(19) United States
(12) Patent Application Publication
(10) Pub. No.: US 2026/0355306 A1
(43) Pub. Date: Nov. 12, 2026

(54) VACCINES FOR HUMAN CYTOMEGALOVIRUS

(71) Applicants: Cornell University, Ithaca, NY (US); Albert-Ludwigs-Universitat Freiburg, Freiburg (DE)

(72) Inventors: Sallie R. Permar, New York, NY (US); Claire E. Ramloul, New York, NY (US); Hartmut Hengel, Freiburg (DE); Philipp Kolb, Freiberg (DE)

(21) Appl. No.: 19/491,650

(22) PCT Filed: Jun. 7, 2024
(86) PCT No.: PCT/US2024/032999

§ 371 Date: Dec. 9, 2025

Related U.S. Application Data
(60) Provisional application No. 63/472,149, filed on Jun. 9, 2023.

(51) Int. Cl. A61K 39/245; A61K 39/00; A61K 39/39; A61P 37/04

(52) U.S. Cl. CPC A61K 39/245; A61K 39/39; A61P 37/04; A61K 2039/55566

(57) ABSTRACT

The present disclosure provides vaccines that include one or more HCMV viral Fc receptors (vFcγRs) or immunogenic fragments thereof and methods related thereto.

### CROSS-REFERENCE TO RELATED APPLICATIONS

This application claims the benefit of U.S. Provisional Application No. 63/472,149, filed on Jun. 9, 2023, the entire contents of which are incorporated herein in their entirety by this reference.

### STATEMENT REGARDING FEDERAL FUNDING

This invention was made with government support under contract number AI129859 awarded by National Institutes of Health. The government has certain rights in the invention.

### FIELD OF INVENTION

The present disclosure relates to vaccine compositions and associated methods for treating and/or preventing human cytomegalovirus (HCMV) infection. The invention particularly relates to vaccine compositions that target a combination of (i) one or more HCMV viral Fc receptors (vFcγRs) and (ii) one or more additional components such as, e.g., glycoprotein B (gB) and pentameric complex (PC).

### BACKGROUND

Congenital cytomegalovirus (cCMV) is the most common congenital infection globally, with approximately 1 in 200 neonates born with CMV in the U.S. cCMV causes an immense global burden of hearing loss and other neurodevelopmental deficits in affected infants. In fact, cCMV is the leading non-genetic cause of hearing loss, accounting for 10-20% of pediatric cases. The frequency of vertical transmission is higher (30-40%) in women who experience primary CMV infection during pregnancy compared to those who experience viral reactivation or re-infection (1-2%), suggesting that the maternal adaptive immune system can partially protect against placental CMV transmission. Thus, prenatal maternal vaccination to induce or boost protective responses is a promising strategy for reducing the incidence of cCMV and associated neurologic impairment. Yet, there is currently no licensed vaccine, in part because vaccines in development have not yet elicited superior protective immunity to that elicited by natural HCMV infection.

### SUMMARY

Cytomegalovirus (CMV) is the most common congenital infection and a problematic opportunistic infection in immunocompromised patient populations. As such, an effective vaccine against CMV has been a top priority in medicine for decades, but a vaccine has yet to be licensed. The gold standard benchmark for a CMV vaccine is an immune response greater than that elicited by natural infection, which has yet to be achieved. A potential reason for the difficulties in developing an effective CMV vaccine is CMV's numerous and potent immune evasion mechanisms. One such mechanism is the expression of multiple IgG binding proteins that have demonstrated the ability to interfere with activation of host Fc receptors, which are critical for antibody mediated cellular immunity. These functions, such as antibody dependent cellular cytotoxicity (ADCC) and phagocytosis (ADCP), have been implicated in protection against CMV disease outcomes, such as congenital infection and viremia following solid organ transplant.

The present disclosure provides the insight that directly targeting CMV immune evasion mechanisms is a potential strategy for improving vaccine efficacy. HCMV encodes three decoy Fc gamma receptors (gp34, gp68, gp95) that can bind preferentially to CMV-specific IgG and interfere with host Fc mediated antibody effector responses. In some embodiments, inclusion of one or more of gp34, gp68, and gp95 in a CMV vaccine improves host mediated antibody effector responses which have been implicated in prevention of adverse CMV disease outcomes. Addition of viral Fc receptors to CMV vaccine antigens may enhance a subject's Fc-mediated antibody functions, and may protect against congenital CMV transmission. In some embodiments, provided are vaccine strategies targeting a combination of HCMV vFcγRs (gp34, gp68, and/or gp95) in addition to key glycoprotein immunogens that have achieved partial success in clinical vaccine trials, such as glycoprotein B (gB) and the pentameric complex (PC), will elicit more effective Fc-mediated antibody effector responses than vaccination with glycoprotein target(s) alone.

### BRIEF DESCRIPTION OF THE DRAWINGS

The Drawing included herein, which is composed of the following Figures, is for illustration purposes only and not for limitation.

FIG. 1 provides a schematic for how vaccination against vFcγRs may reduce the risk of congenital CMV infection. As depicted, vaccine-elicited vFcγR-targeting antibodies may block vFcγR-Fc binding and reduce vFcγR-mediated inhibition of Fc-mediated effector responses. Relieving this inhibition may improve host FcγR mediated cellular immunity and potentially prevent vertical CMV transmission.

FIG. 2 provides an exemplary rabbit vaccination and sampling schedule. Young rabbits were vaccinated thrice, each dose 4 weeks apart, with an Addavax-adjuvanted protein subunit vaccine composed of gB alone or in combination with one of the HCMV vFcγRs.

FIG. 3 provides a graph of vaccine-elicited gB antibody binding responses as measured by ELISA. The kinetics and magnitude of gB-binding IgG titers were remarkably consistent among the vaccine groups.

FIG. 4 provides graphs of gB IgG binding responses and FcγR binding as measured by BAMA. Breakdown of the gB binding response representing the full-length vaccine antigen, only the ectodomain of gB, and the cytosolic antigenic domains (ADs). Results are shown for total IgG binding to the gB measured using an anti-rabbit IgG-PE secondary antibody as well as FcγRI (CD64) and FcγRIII (CD16) binding to gB-specific IgG using biotinylated FcγRs followed by streptavidin-PE for fluorescent detection of binding.

FIG. 5 provides a graph of CD64 Activation at week 10 Post-Prime. CD64-expressing IL-2 reporter cells were used to evaluate CD64 activation by plasma antibodies targeting AD169r infected cells. Data is reported as the raw OD₄₅₀ from an IL-2 detection ELISA of undiluted culture supernatant.

FIG. 6 provides a graph of ADCP Responses to Co-immunization with gB and Individual vFcγRs. The ability of vaccine elicited plasma antibody responses to mediate ADCP against free virus was evaluated longitudinally. The co-immunizations all demonstrated higher ADCP from Week 10 on over gB alone, with gp34 vaccination yielding the greatest improvement.

FIG. 7 provides a graph of viral load as measured by qPCR detection of RhCMV 1E-1 in plasma following primary infection of seronegative rhesus macaques. Control of viremia was defined as the first time point with undetectable viral load. Selection of vFcγRs led to more rapid RhCMV control.

FIG. 8 provides graphs depicting antibody responses elicited by gB/Addavax in rabbits (n−6). IgG binding to gB measured by (A) ELISA and (B) transfected cell binding assay. (C) Neutralization of AD169r-GFP HCMV on fibroblasts (MRC-5) and epithelial cells (ARPE), with or without complement (+/−C). (D) ADCC. (E) ADCP. (F) Fc gamma receptor (FcgR) binding to gB-specific plasma antibodies measured by binding antibody multiplex assay. Dashed horizontal lines represent the assay limit of detection (LOD) or average of seronegatives (week 0). (G) gB-specific T cell responses measured by intracellular cytokine staining for IFNγ following stimulation by no peptide (NS), ConA (positive control), or gB peptides.

FIG. 9 depicts a graph showing that a subset of mAbs generated against gp34 and gp68 can rescue CD16 activation. Target cells were co-transfected with HER2 antigen and the vFcγR of interest and then incubated with HER2-specific IgG (Herceptin) and the identified mouse mAbs, either as Fab2 fragments (gp34) or whole mAb molecules (gp68). CD16 activation was quantified using IL-2 reporter cells expressing the human CD16 receptor with the CD3 intracellular signaling domain, reporting the optical density from an IL-2 detection ELISA of culture supernatant. The upper range of activation was determined using target cells only expressing HER2 antigen (black bar), and the maximal inhibition by gp34 or gp68 was determined using no antibody (PBS) or non-targeting antibodies (white bars).

FIG. 10A depicts an exemplary vaccination schedule used in the context of the present disclosure.

FIG. 10B depicts step-wise antigen selection process used in the context of the present disclosure.

FIG. 10C depicts exemplary criteria for antigen.

FIG. 11 depicts graphs of plasma antibody responses in rabbits immunized with gB alone or with one of the vFcγRs (n=4 per group). Measured by ELISA, reporting AUC of OD450 over 1:30-65,610 dilution series (log). His-tagged gp34mtrp (lacking Fc binding) was used as a preliminary assessment of anti-vFcγR responses.

FIG. 12 depicts a schematic of a Binding Antibody Multiplex Assay.

FIG. 13A-FIG. 13F show that vFcγRs interfere with host FcγR activation against rabbit and human IgG.

FIG. 14A-FIG. 14C depict graphs showing the vFcγR-binding responses. In FIG. 14A, the 20 μg dose group (Freiburg antigen) on the graph is marked with a symbol of solid diamond and open circle. The 40 μg dose group (Freiburg antigen) is marked with a symbol of open triangle and open square. The gB only vaccine group is marked with a symbol of inverted triangle. The remaining symbols represent the 20 μg dose group (in-house antigen). In FIG. 14B and FIG. 14C, the 40 μg dose group (Freiburg antigen) is marked with a symbol of solid diamond and open circle. The gB only vaccine group is marked with a symbol of open triangle and open square. The remaining symbols represent the 20 μg dose group (in-house antigen).

FIG. 15A-FIG. 15D show that inclusion of HCMV vFcγRs at 40 μg dose in gB protein subunit vaccine improves ADCP function broadly across several HCMV isolates.

FIG. 16 shows the ADCC function detectable in a greater number of animals immunized with vFcγRs compared to gB alone.

FIG. 17 depicts a schematic showing that vFcγRs are not redundant. Adapted from Kolb et al. (eLife 2021 doi:10.7554/eLife.63877), which is incorporated herein by reference.

### DETAILED DESCRIPTION

Congenital cytomegalovirus (cCMV) is the most common in utero infection affecting approximately 1 in every 200 newborns and causing devastating neurologic impairment in approximately 1 in 5 infected infants. Placental transmission of CMV is significantly reduced in chronically infected women (1-2%) compared to those who experience primary CMV infection during pregnancy (30-40%), suggesting that the CMV-specific maternal adaptive immunity can protect against cCMV infection. Thus, vaccination is a promising strategy for preventing cCMV and reducing the burden of CMV-associated neurologic impairment. However, there is currently no licensed vaccine to prevent cCMV infection. Several challenges have limited CMV vaccine development, including limited animal models due to high species-specificity and numerous viral immune evasion mechanisms.

The most efficacious CMV vaccine to date is a HCMV glycoprotein B (gB) subunit adjuvanted with MF59, which achieved ~50% efficacy against viral acquisition in multiple trials. This vaccine elicited a low-level neutralization response, but robust non-neutralizing antibody responses, such as antibody dependent cellular phagocytosis (ADCP), likely contributed to the partial protection observed.

The present disclosure proposes targeting CMV evasins as a strategy to boost Fc-mediated antibody effector responses. The present disclosure encompasses a recognition that Fc-mediated effector functions are vital anti-CMV responses. This is supported by a recent study by the present inventors that identified that high maternal ADCP responses were associated with decreased risk of cCMV transmission in a cohort of CMV-seropositive mothers and their infants. This finding was further supported by robust maternal plasma HCMV-specific IgG CD64 (FcγRI) and CD32A (FcγRIIA) engagement profile.

The present disclosure describes experiments in a rhesus macaque (RM) model of primary maternal CMV infection, which suggests that pre-existing natural killer (NK) cell mediated antibody dependent cellular cytotoxicity (ADCC) is an important contributor to reducing vertical CMV transmission risk. Collectively, this work reveals that Fc-mediated antibody functions may protect against cCMV.

CMV is adept at immune evasion. A large proportion of CMV's genome encodes immune evasin proteins, including an array of decoy cytokines and immune receptors. Human CMV (HCMV) expresses three glycoproteins termed viral Fc receptors (vFcγRs) capable of binding the Fc region of immunoglobulin G (IgG) and have been shown in vitro to interfere with host Fc-mediated antibody effector functions, which have been identified as important maternal immune factors for prevention of cCMV transmission. Specifically, HMV expresses three glycoproteins with immunoglobulin (Ig)-binding ability that interfere with host FcγR function (gp34, gp68, gp95) but are distinct from host FcγRs. Unlike host FcγRs, these viral Fcγ receptors (vFcγRs) have demonstrated binding to multiple subclasses of IgG in a glycan independent manner. When expressed on the surface of infected cells, modulation of host IgG activity can occur by several potential mechanisms, including blocking host FcγR activity by bipolar bridging of HCMV-specific IgG bound to antigen expressed on infected cells and vFcγR-mediated internalization and degradation of host IgG by infected cells. Current evidence suggests that gp34 and gp68 can bind IgG simultaneously at different epitopes of the Fc region. A synergistic impairment of host FcγRIII-mediated responses by these molecules has been described, as well as the ability of gp68 to block host FcγR interaction with IgG immune complexes and of gp34 to facilitate IgG internalization. Thus, this suite of vFcγR proteins helps facilitate CMV's ability to evade humoral immunity.

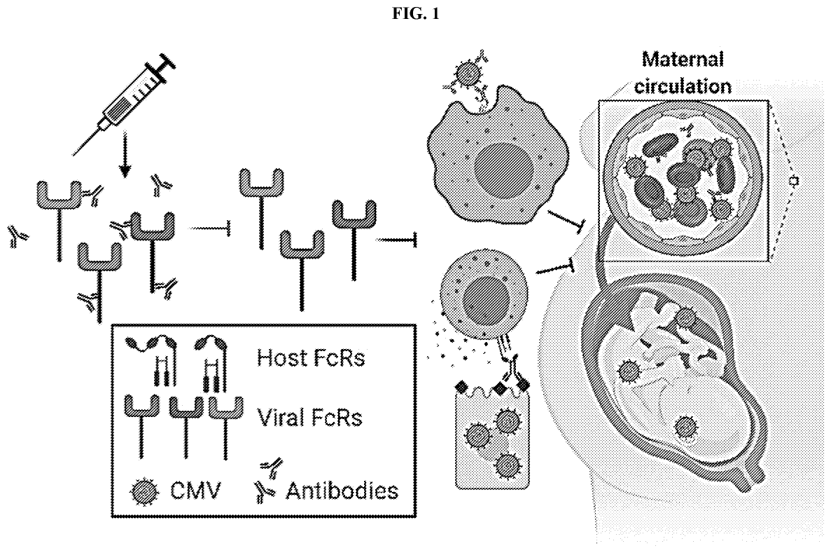

Antibody responses mediated by FcγRs have been implicated in protection against vertical CMV transmission. For example, antibodies elicited against vFcγRs may block this immune evasion mechanism, allowing maternal antibody effector responses to be significantly more effective due to lack of vFcγR inhibition. Without wishing to be bound by theory, it is proposed that antibodies against vFcγRs may prevent their inhibition of host Fc-mediated antibody effector responses, as depicted in FIG. 1, thereby improving potentially protective Fc-mediated antibody effector functions.

Current CMV vaccines include viral entry proteins, but have yet to incorporation targets of immune evasion, which may restore the potency of responses against common immunogens. Therefore, this is a novel vaccine strategy against a pathogen for which natural immunity is not protective.

The present disclosure describes experiments to determine if active vaccination against HCMV vFcγRs using an adjuvanted protein subunit platform can elicit antibodies against vFcγRs. The examples below provide evidence to suggest that active vaccination against HCMV vFcγRs improves activation of host FcγRs.

This pre-clinical evaluation of vFcγR-targeting vaccine strategies will provide proof-of-concept evidence regarding the impact of vFcγR active vaccination on the function of vaccine elicited responses against other antigens, potentially providing a simple modification to partially efficacious vaccines in development (e.g. gB/MF59, mRNA1647).

Leveraging lessons from natural CMV immunity and prior partially-effective vaccines to de-risk CMV vaccine development and future human efficacy trials. As there are 3 functional vFcγRs encoded in the HCMV genome (gp34, gp68, and gp95) yielding 3potential new vaccine antigens, it will be important to define the vFcγR antigen(s) that will be most impactful in improving the vaccine-elicited functional antibody response. This pre-clinical evaluation of immunogenicity will serve to de-risk costly nonhuman primate (NHP) challenge studies and clinical studies by narrowing the number of vaccine candidates, focusing on those that best elicit potentially-protective immune response.

A viral FcR antigen-targeting approach has been successful in enhancing VSV immunity to prevent Shingles (Shingrix).

Experiments to assess immunogenicity of vFcγRs in natural HCMV infection, determine the optimal combination of vFcγRs and other glycoprotein vaccine antigens, such as gB and the pentameric complex (PC), and maximize vaccine-elicited Fc-mediated effector responses using a rabbit immunogenicity model are also described.

In some embodiments, the present disclosure provides HCMV vaccine compositions comprising one or more polypeptide antigens selected from gp34, gp68, and gp95. In some embodiments, further truncation or modification of the proteins may be used to better expose the active site or antibody binding epitopes. In some embodiments, vaccination using the linear peptide of binding epitope(s) is an additional option for specifically eliciting responses against the region where antibodies block vFcγR function.

In some embodiments, vaccine compositions described herein may be useful in a population for prevention of congenital CMV. In some embodiments, a population for vaccination to prevent congenital CMV may include females of child-bearing age. In some embodiments, vaccination is administered prior to conception with durable anti-vFcγR antibodies through the entire pregnancy. In some embodiments, vaccination is administered in the first trimester, with durable anti-vFcγR antibodies through the remainder of the pregnancy.

The present disclosure encompasses a recognition that such vaccines may also be useful in other populations at risk for severe CMV disease, such as cancer patients, solid organ transplant recipients, and people living with uncontrolled HIV.

In some embodiments, vFcγRs antigens for use in the context of the present disclosure may be produced using methods known in the art. For example, in some embodiments, such methods include transfection of expression plasmid of His tagged proteins in mammalian 293F cells followed by Nickel bead purification and subsequent HPLC. Current pre-clinical vaccine regimens have included a 3-dose series of 20 μg per protein, each one month apart.

In some embodiments, mammalian cell transfection may be used for proper glycosylation. In some embodiments, nickel bead purification is sufficient to isolate His tagged proteins and HPLC ensures that the proteins included in the vaccine are a single molecular species are not aggregated or fragmented. In some embodiments, other methods of achieving high purity protein may also be used.

The examples below leverage systems immunology in an HCMV vaccine immunogenicity model informed by partially-protective HCMV vaccines. Multiplexed antibody binding measures are combined with polyfunctional antibody testing and polyfunctional T cell measures in the rabbit immunogenicity model to compare both specificity and function of vaccine-elicited immunity benchmark the novel vaccine approach to that of the clinically-tested and partially-effective gB/MF59 vaccine.

### Exemplary Embodiments

1. An immunogenic composition comprising one or more cytomegalovirus (CMV) vFcγRs antigens comprising gp34, gp68, gp95, or an immunogenic fragment of any thereof.
2. A combination comprising two or more cytomegalovirus (CMV) vFcγRs antigens comprising gp34, gp68, gp95, or an immunogenic fragment of any thereof.
3. A combination comprising: (a) one or more cytomegalovirus (CMV) vFcγRs antigens or a nucleic acid encoding the same, and (b) one or more additional CMV antigens or a nucleic acid encoding the same, wherein the vFcγRs antigens comprise gp34, gp68, gp95, or an immunogenic fragment of any thereof, and wherein the additional CMV antigens are different from the vFcγRs antigens.
4. The combination of 3, wherein the one or more additional CMV antigens comprise glycoprotein B (gB), pentameric complex (PC), pp65, immediate-early protein-1 (IE-1), or an immunogenic fragment of any thereof.
5. The combination of any one of 2-4, wherein the one or more CMV vFcγRs antigens are polypeptides.
6. The combination of any one of 3-5, wherein the one or more additional CMV antigens are polypeptides.
7. The combination of any one of 2-4, comprising a nucleic acid encoding the one or more CMV vFcγRs antigens.
8. The combination of 3, 4, 6, or 7, comprising a nucleic acid encoding the one or more additional CMV antigens.
9. The combination of any one of 2-8, wherein the one or more CMV vFcγRs antigens comprise gp34 or an immunogenic fragment thereof.
10. The combination of 9, wherein the one or more CMV vFcγRs antigens further gp68 or an immunogenic fragment thereof.
11. The combination of 9 or 10, wherein the one or more CMV vFcγRs antigens further gp95 or an immunogenic fragment thereof.
12. The combination of any one of 2-11, wherein the one or more CMV vFcγRs antigens can elicit an antibody response that blocks vFcγR binding to IgG Fc in a subject.
13. The combination of any one of 3-12, wherein the one or more additional CMV antigens comprise (i) gB or an immunogenic fragment thereof and (ii) PC or an immunogenic fragment thereof.
14. A combination comprising a gp34 polypeptide or an immunogenic fragment thereof and a glycoprotein B (gB) polypeptide or an immunogenic fragment thereof.
15. The combination of 14, further comprising a gp68 polypeptide or an immunogenic fragment thereof.
16. The combination of 14 or 15, further comprising a gp95 polypeptide or an immunogenic fragment thereof.
17. A vaccine comprising the immunogenic composition of 1 or the combination of any one of 2-16.
18. The vaccine of 17, further comprising an adjuvant.
19. The vaccine of 18, wherein the adjuvant is MF 59, Addavax, or other squalene emulsion adjuvant.
20. A pharmaceutical composition comprising the combinations of any one of 2-16.
21. A method comprising administering one or more doses of the combination of any one of 2-16 to a subject.
22. The method of 21, wherein the method induces an immune response in a subject.
23. The method of 21 or 22, wherein the subject is a female of child-bearing age.
24. The method of 21 or 22, wherein the subject is at risk for severe CMV disease.
25. The method of 24, wherein the subject has cancer, HIV, and/or has received a solid organ transplant.
26. A method of any one of 21-25, wherein the administering generates anti-CMV antibodies in a subject.
27. The method of any one of 21-26, wherein the administering has improved Fc-mediated antibody effector response to the one or more additional CMV antigens, relative to a subject that has been administered only the one or more additional CMV antigens.
28. The method of any one of 21-27, wherein the administering has increased CD64 activation, increased CD16 activation, and/or increased CD32 activation, relative to a subject that has been administered only the one or more additional CMV antigens.
29. The method of any one of 21-28, wherein the administering has increased antibody dependent cellular cytotoxicity (ADCC) and/or increased antibody-dependent cellular phagocytosis (ADCP) response, relative to a subject that has been administered only the one or more additional CMV antigens.

### EXEMPLIFICATION

### Example 1: Pre-Clinical Testing of HCMV vFcγRs as Additional Vaccine Antigens

Vaccine prevention of cCMV is a critical strategy for reducing the high burden of CMV-associated neurologic impairment in newborns globally. The present example describes pre-clinical testing of HCMV vFcγRs as additional vaccine antigens.

Previous work from our group has implicated Fc mediated effector antibody responses in prevention of vertical CMV transmission. The present disclosure encompasses a recognition that Fc mediated antibody effector functions may have a role in preventing congenital CMV. RhCMV encodes three vFcγRs that appear to function in evasion of these key Fc-mediated antibody effector functions, based on both in vitro and in vivo studies. In the absence of vFcγRs, RhCMV can be controlled more quickly in vivo, which suggests that an intervention targeting vFcγRs may be able to reduce the risk of vertical CMV transmission through 1) reduction in the duration of viremia and/or 2) improvement in Fc mediated effector functions.

Targeting immune evasion mechanisms may be a promising strategy for achieving the gold standard benchmark in CMV vaccine development of exceeding infection-elicited immunity, since this is only partially protective against vertical CMV transmission. The most efficacious vaccine to date was a gB subunit with MF59, a squalene emulsion adjuvant, achieving about 50% efficacy against primary CMV acquisition in two clinical trials in women of child-bearing age. The objective of the current study was to demonstrate proof-of-concept that targeting HCMV vFcγRs by active vaccination in addition to other immunogenic CMV antigens could be a promising strategy for improving CMV vaccines in development. The present example utilized a rabbit immunogenicity model to evaluate the addition of individual vFcγRs to a gB protein subunit vaccine adjuvanted with the squalene emulsion Addavax, modeling the gB/MF59 vaccine. It was hypothesized that addition of vFcγRs to a CMV vaccine would elicit antibody responses against the vFcγRs capable of mitigating their immune evasive function and yield greater Fc mediated antibody effector function than the original vaccine. Given the implication of these antibody functions in prevention of vertical CMV transmission, such an effect may ultimately reduce the risk of congenital CMV, see FIG. 1.

### 1.1 Rabbit Vaccination Schedule

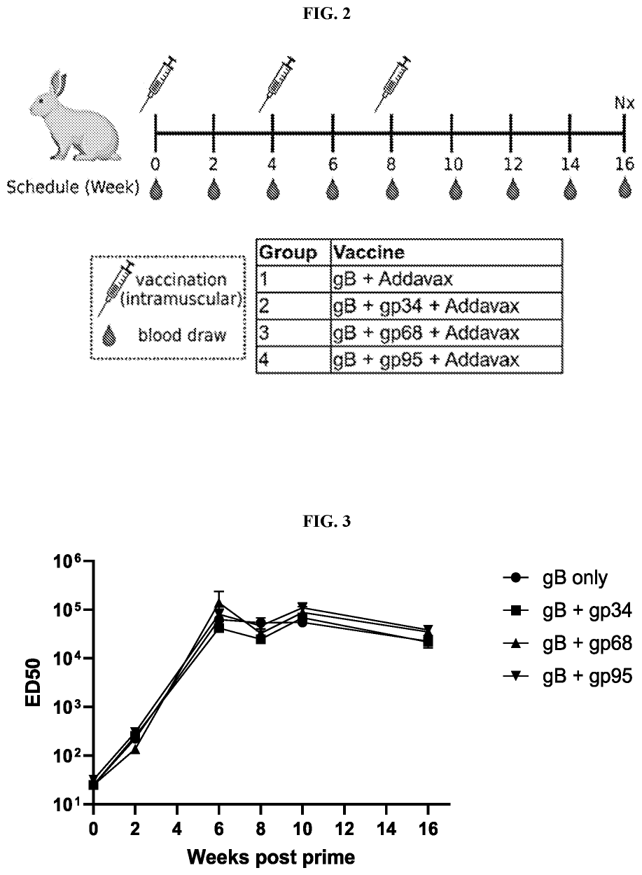

To model the gB/MF59 vaccine, a full length gB protein subunit lacking only the transmembrane domain adjuvanted with Addavax, a bioequivalent of the proprietary MF59 squalene emulsion adjuvant. Animals were vaccinated with gB/Addavax alone or in combination with one of the three functional vFcγRs (n=4 per group), also included as protein subunits lacking transmembrane domains. gpRL13 was not included in this study as it has not demonstrated inhibition of host FcγR activation in our hands, possibly due to the short duration of expression on the cell membrane due to a high turnover rate. Vaccines were administered intramuscularly in a three-dose series, each one month apart. Blood was collected every two weeks for 16 weeks, FIG. 2.

1.2 Addition of vFcγRs to gB/Addavax Vaccine Does Not Affect Vaccine-Elicited gB Binding Antibody Responses

gB-specific antibody binding responses were evaluated via ELISA. The kinetics of the humoral response against gB was very similar among the four vaccine groups, FIG. 3. This was unsurprising since the inclusion of the vFcγRs should not impact components of the humoral response against gB that do not involve host FcγRs (i.e., gB-specific binding and neutralization).

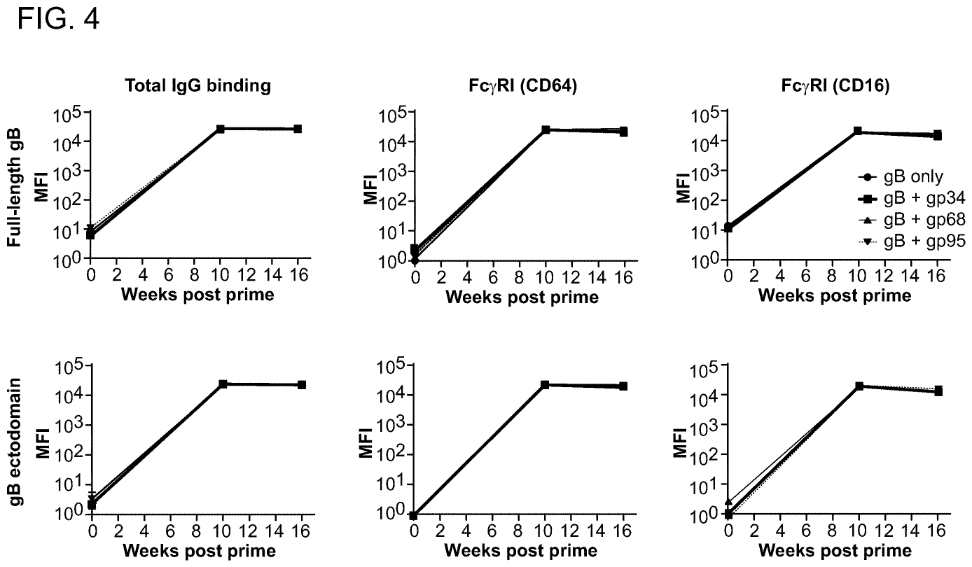

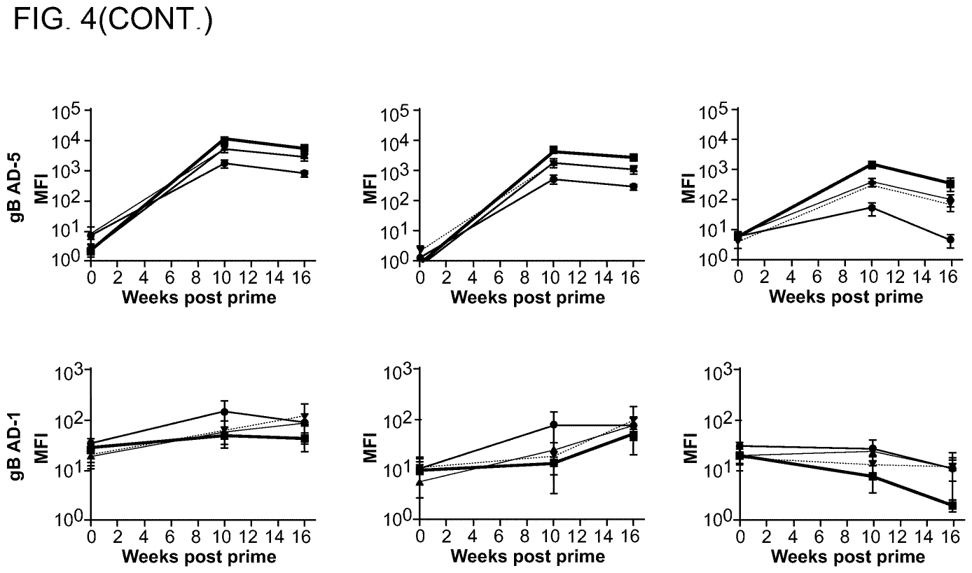

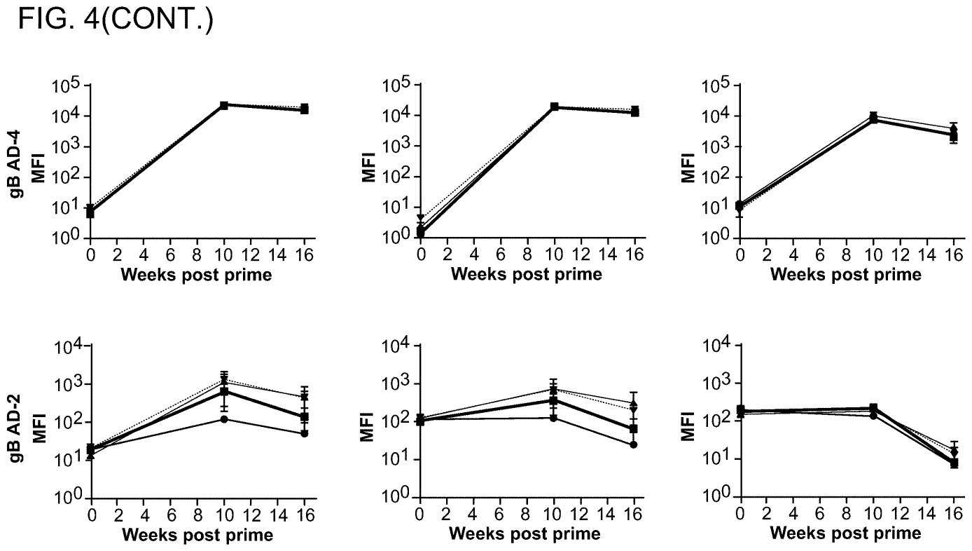

IgG binding responses against gB and individual antigenic domains (ADs) were also evaluated using a semi-quantitative binding antibody multiplex assay (BAMA). IgG binding to the full-length gB (vaccine antigen), the ectodomain, and AD-4 were very consistent among the four groups. There was greater variation among the groups in binding against AD-1, AD-5, and AD-2, although binding was notably low against AD-1 and AD-2, FIG. 4.

FcγRI (CD64) and FcγRIII (CD16) binding to vaccine elicited gB-specific IgG were also assessed using the same antigen panel, FIG. 4. FcγR binding depends greatly on total IgG binding, and the kinetics of FcγR binding closely follow the same pattern as the total IgG binding for both CD64 and CD16. Only relatively small differences in the total IgG binding and FcγR binding with AD-1, AD-2, and AD-5 were observed between vaccine groups.

1.3 Co-Immunization With gB and gp34 Yields Greater CD64 Activation and ADCP Than gB Alone

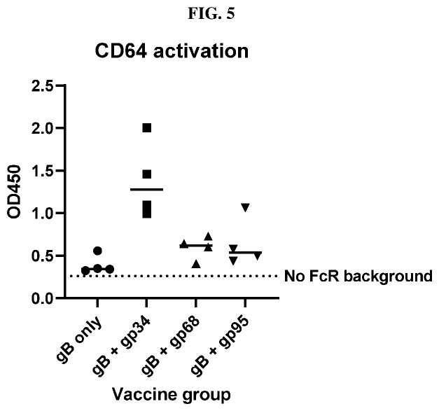

Using a IL-2 reporter assay, CD64 activation was assessed using plasma samples from the week 10 time point (2 weeks after the last boost), which was expected to be the peak humoral response. A clear increase in CD64 activation was observed in all four rabbits vaccinated with gB and gp34 over animals vaccinated with gB alone, FIG. 5. While there was a marginal increase in CD64 activation over gB alone in rabbits immunized additionally with gp68 and gp95, the biological significance of such a small change is not clear.

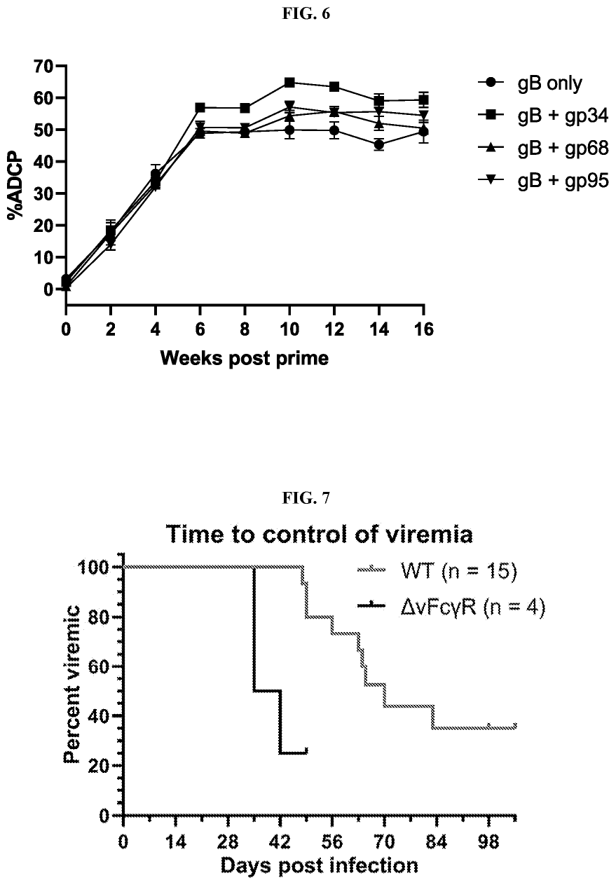

Next, the functional consequences of the observed differences in CD64 activation were evaluated by measuring ADCP function. The finding that gB/Addavax elicits robust ADCP responses were consistent with previous work in the rabbit immunogenicity model. Interestingly, all three co-immunization strategies yielded greater ADCP responses over gB alone from weeks 10-14 post prime, FIG. 6. Following a similar pattern to the CD64 activation result, it was observed that the greatest improvement in the gp34 co-immunized group and a smaller effect size in the gp68 and gp95 co-immunized groups.

### 1.4 Conclusions

The results of this study demonstrate that vaccination against vFcγRs can elicit immune responses capable of counteracting this immune evasion mechanism and improving functional Fc mediated effector responses. Notably, the improvement observed in CD64 activation was not a result of greater CD64 binding to gB-specific antibodies as gp34 co-vaccination only resulted in slightly higher CD64 binding to gB AD-5 in our BAMA assay.

It is important to note that ADCP can be mediated by both CD64 and CD32A. The THP-1 cells that were utilized for this assay express both receptors, but CD64 is expressed at a higher level than CD32A. Thus, the greater separation observed between the gB-only vaccine group and the gp68 and gp95 co-immunization groups may be a result of a compounded effect. This will be further evaluated by measuring CD32A activation as described herein for CD64.

Another key difference between the CD64 activation assay and the functional ADCP assay is the target, as the FcγR activation assay uses infected target cells while the ADCP assay targets free virus. Thus, an additional inference that can be made from this result is that the vFcγRs function on the surface of the virion as they do on infected cells because if not, highly similar ADCP responses, similar to the gB binding results, would be expected.

These results regarding CD64 activation support that the addition of a single vFcγR to a vaccine similar to the gB/MF59 vaccine could improve FcγR activation and functional Fc mediated antibody effector responses. Given the implication of Fc mediated effector responses in protection from vertical CMV transmission in human observational studies, further evaluation of this concept is proposed in the rhesus macaque model where efficacy can be evaluated in the context of congenital infection.

The preliminary evidence from this study suggests that vaccination with gp34 elicits the strongest effect on improving host FcγR activation. Additionally, combinations of antigens may be used to optimize a response. Combinations of vFcγRs along with common vaccine antigens, gB and pentamer, will be assessed. The present disclosure recognizes that a combination of gB and pentamer will elicit stronger ADCC responses at baseline since gB/Addavax has demonstrated very limited ADCC responses in a previous study in the rabbit model. Stronger baseline function may allow for further improvement through inclusion of one or more vFcγRs. High performance liquid chromatography will be used to produce high purity proteins for use in any future vaccine studies, including dose optimization, as simple affinity chromatography targeting the histidine tag on the proteins may yield products with minor impurities.

Additional studies are also proposed to evaluate antibody binding and T cell responses to the vFcγRs. Since the vFcγRs bind to the Fc region of IgG, the antigen binding fragment (Fab) from IgG can be purified from the vaccinated rabbits to evaluate vFcγR-targeting responses. FcγR activation may be further assessed by CD16 and CD32 activation as described above for CD64. Antibody ADCC function can be assessed by neutralization assays and NK cell degranulation assays. The breadth of antibody responses may be evaluated by FcγR activation against target cells infected with a panel of HCMV strains with variation in each of the vFcγR proteins.

### 1.5 Methodology

### Protein Production and Vaccine Formulation

Histidine-tagged, codon-optimized RL11 (gp34), RL12 (gp95), and UL119/118 (gp68) from the AD169r strain of HCMV, all lacking transmembrane domains were each expressed using a pIRES-eGFP plasmid vector. These plasmids were a gift from co-investigators Philipp Kolb and Hartmut Hengel. Each of the vFcγR proteins were produced by transient transfection in mammalian 293F cells using the ExpiFectamine 293 transfection kit (Gibco) according to manufacturer instructions. After 5 days, the cell supernatant was harvested, and each protein was purified using Nickel bead chromatography targeting the histidine tag. Protein concentration was quantified using a Bradford assay using bovine serum albumin to derive a standard curve for interpolation. Western blots and Coomassie-stained SDS-PAGE gels were performed as quality control measures for proper protein expression. Endotoxin levels were confirmed to be below the acceptable level before incorporation into vaccine formulations. The HCMV gB used in vaccine formulations and assays was a full-length protein lacking only the transmembrane domain, purchased from Sino Biological (cat. 10202-VCCH1). Notably, this protein does not contain a histidine tag. All proteins were stored at −80 °C until use.

Vaccines were comprised of 20 μg of gB alone or with 20 μg of gp34, gp68, or gp95 (for 4 total vaccine groups) diluted to 250 μL in sterile PBS. The protein mix was then combined 1:1 with the squalene emulsion adjuvant Addavax (InvivoGen).

### Rabbit Vaccination and Sample Collection

Two month old New Zealand White rabbits were purchased from Charles River Laboratories (Wilmington, MA) and housed at Weill Cornell Medicine. For blood collection and vaccinations, rabbits were sedated with 20-50 mg/kg of ketamine and 1-4 mg/kg of midazolam intramuscularly (IM) in epaxial muscles. Glycoprrolate (0.01-0.02 mg/kg) was used as a pre-anesthetic and maropitant (2 mg/kg) as an analgesic. Isoflurane inhalant (0.6-2.5%) was used by nose cone to maintain anesthesia. The neck was shaved, and lidocaine cream was applied prior to blood draw. Blood was collected by jugular vein into an EDTA-coated collection tube. Immediately after blood collection, the vaccine was administered intramuscularly in the epaxial muscle opposite used for sedation.

Blood was processed via a standard procedure as previously described¹⁰³ Briefly, whole blood was spun down by low-speed centrifugation to separate the plasma and cells. The plasma layer was collected and spun down again at 2000 rpm for 15 minutes to pellet any cellular contamination. Plasma aliquots were stored at −80 °C until further use. Peripheral blood mononuclear cells were isolated from the buffy coat layer via density gradient (CedarLane Lympholyte cat. CL5120), red blood cells were removed using ACK Lysing buffer (Gibco), and cells were stored in liquid nitrogen until further use.

### Virus Growth

Stocks of AD169r-GFP was produced by propagating from a seed stock on ARPE cells for 2-4 passages and then once of HFF- 1 cells to boost viral titer. Infection was performed in a low volume of reduced-FBS medium for at least 2 hours at 37 °C and 5% CO₂, a maintenance volume of media was added, and cells were incubated for up to 2 weeks. Virus was harvested when 90% cytopathic effect was observed. Cells were collected by scraping and combined with the culture supernatant. Cells were pelleted by low-speed centrifugation and supernatant collected and placed on ice. Cell pellets were resuspended in infection media and combined, then subjected to either three freeze/thaw cycles or sonication. Large cell debris was pelleted by low-speed centrifugation. The supernatants were combined, passed through a 0.45-μm filter, overlaid onto a 20% sucrose cushion, and then ultracentrifuged at 70,000 g for 2 hours at 4 °C using an SW28 Beckman Coulter rotor. Virus pellets were resuspended in DMEM containing 10% sucrose and titered on HFF-1 and/or ARPE cells (depending on the intended use) using the TCID₅₀ method.

### Antibody Binding Assays

IgG binding to gB was first assessed by ELISA. High-binding clear 384-well plates (Corning 3700) were coated with 2 μg/mL HCMV gB (Sino Biological) in 0.1M sodium bicarbonate (pH=9.55) overnight at 4 °C. Following overnight incubation, plates were blocked for 1-2 hours with blocking solution (PBS+, 4% whey protein, 15% goat serum, 0.5% Tween-20), and then 6-fold serial dilutions of plasma (1:50 to 1:13,996,800) were added to the wells in duplicate for 1-2 hours. Plates were then washed twice using an automated plate washer (BioTek) and incubated for 1 hour with polyclonal goat anti-rabbit IgG (H+L) HRP-conjugated secondary antibody (Southern Biotech) at a 1:5,000 dilution. After four washes, SureBlue Reserve TMB Microwell Peroxidase Substrate (KPL) was added to the wells for 3.5 minutes, and the reaction was stopped by addition of an equal volume of 1% HCl solution (KPL). Plates were read at 450 nm.

CMV proteins were conjugated to BioRad magplex magnetic COOH beads using an EDC and NHS coupling reaction (BioRad). Two panels were run. Proteins included in the first panel were HCMV post-fusion gB (full-length) and gB ectodomain. Proteins included in the second panel were gB AD-5/Domain I, gB AD-1, gB AD-4/Domain II, and gB AD-2 site 1. For plates containing the gB panel, rabbit plasma samples were diluted 1:250 in diluent (1×PBS, 1% Non-Fat Dry Milk Powder 5% Normal Goat Serum, 0.05% Tween-20) and plated in duplicate. For the second panel, Rabbit serum samples were diluted 1:100 in diluent and plated in duplicate. Samples were incubated with beads for 30 minutes while shaking at 180 rpm at room temperature. FcγRIA-biotin, FcγR3A-biotin, and anti-rabbit IgG-PE secondary antibodies were diluted to 2 μg/mL in BAMA diluent and Fcγ receptors were incubated with streptavidin-PE (Southern BioTech Cat. No. 7105-09M) for at least 10 minutes. FcγR1A-PE was incubated with samples for 3 hours rotating 180rpm at room temperature. Fcγr3a-PE and anti-Rabbit IgG-PE incubated with samples for 30 minutes. Plates were washed three times and read on the BioRad BioPlex 200 instrument. Results were reported as mean fluorescence intensity (MFI). To control for background binding, a blank well of conjugated beads diluted in BAMA diluent was included on each plate and used to background subtract for the corresponding bead in the sample wells. Additionally, an unconjugated ‘blank’ bead was included in each well as a quality control measure for nonspecific binding.

### CD64 Activation

CD64 activation was measured as previously described¹⁰⁰. Briefly, HFF-1 cells were plated in a culture-treated 96-well flat bottom plate at 5×10⁴ cells per well and incubated overnight to allow cells to adhere. Cells were then infected at 1 MOI with AD169r-GFP for 24 h. Rabbit plasma was diluted 1:25 in HFF-1 culture media and incubated with the infected cells for 3 h. 5×10⁴ BW5147 IL-2 reporter cells expressing CD64-CD3ζ were added for a 1:1 ratio of target to effector cells to each well and incubated for 24 h. IL-2 secretion by the reporter cells was evaluated by ELISA of undiluted culture supernatant and reported as the OD_{450.} Parental BW5147 cells were run in parallel to determine background level IL-2 secretion.

### ADCP

ADCP was assessed by conjugation of concentrated AD169r to AF647 using NHS-ester reaction (Invitrogen), which was allowed to proceed in the dark with constant agitation for 2 hours and then quenched by the addition of pH 8.0 Tris hydrochloric acid, and 250 pfu of the conjugated virus of virus and plasma samples in duplicate at a 1:30 dilution were combined in equal volume (10 μL each) in a 96-well U-bottom plate (Corning) and incubated at 37 °C for 2 hours. THP-1 monocytes were then added at 50,000 cells per well. Plates were spun for 1 hour at 1200×g at 4 °C and then transferred to a 37 °C incubator for an additional 1 hour. Cells were then washed and stained with aqua live/dead (Invitrogen) at 1:1000 for 20 minutes. Following another wash step, cells were fixed for 20 minutes in 10% formalin and resuspended in PBS for acquisition on a BD Fortessa flow cytometer, and data is reported as the percent of the live (aqua-negative) population that was AF647-positive.

### Example 2: RM Infection With Rhesus CMV (RhCMV) Deleted of vFcγRs Results in Rapid Containment of Viremia

The present example describes recent data from the rhesus macaque (RM) model of CMV infection, and also a potential future challenge model to test the vFcγR vaccine concept. The RM model demonstrated that viremia with RhCMV lacking known vFcγRs is contained more quickly than vFcγR-intact virus, FIG. 7. Yet, humoral responses are similar between RMs infected with WT and vFcγR-deleted RhCMV. This in vivo data suggests that adaptive immunity is more effective without interference of vFcγRs in the viral genome, supporting targeting vFcγRs in HCMV vaccines for rapid containment of HCMV viremia after infection.

### Example 3: Defining the Immunogenicity of vFcγRs in Natural HCMV Infection

The present example describes assessment of B and T cell responses that develop against vFcγRs during natural infection. To assess the humoral immunogenicity of each vFcγR, CMV-specific hyperimmune globulin IgG (Cytogam) will be digested at the hinge region and Fab- and Fc purified. Purification may be done protein G purification. If Protein G purification results in incomplete separation of Fab and Fc, alternative chromatography approaches may be used, such as ion exchange, or gel filtration, until each preparation contains only one detectable molecular species. Detection assays may be used to assess binding of each IgG component to each vFcγR expressed on the cell membrane. Additionally, we will stimulate peripheral blood mononuclear cells from CMV-seropositive donors with a peptide pool from vFcγR amino acid sequences and utilize intracellular cytokine staining (ICS) to assess naturally elicited T cell responses against HCMV vFcγRs.

gB and PC are well-known immunodominant glycoproteins, drawing a large proportion of the antibody responses, but antibodies also develop against non-structural proteins, like pp65 and IE-1. T cell responses often target the immediate-early (IE) proteins, possibly due to the immune system seeing those proteins first during periods of viral reactivation, so it is reasonable to expect vFcγRs to also be targeted by such responses.

3.1 Defining the Magnitude and Frequency of IgG Responses Against HCMV vFcγRs in HCMV-Seropositive Individuals.

To assess the natural immunogenicity of each vFcγR, we will transfect 293T cells with gp34-, gp68-, or gp95-, and GFP-expressing plasmid and assess IgG binding to the vFcγRs using cell staining and fluorescence imaging. Successful vFcγR expression will be confirmed by vFcγR binding to purified IgG Fc and a monoclonal antibody Fc as positive controls. To ensure that any observed binding results from vFcγR-targeting antibodies and not vFcγR binding to the Fc region of non-specific antibodies, we will add only the purified Fab fragments from papain-digested IgG purified from HCMV-seropositive individuals (Cytogam) to the transfected cells. We will also utilize Fab from papain-digested IgG purified from serum of seronegative individuals as a negative control. Fab binding under all conditions will be assessed using an anti-Fab primary antibody. Normalization for GFP expression will be performed to control for transfection efficiency. There are two different conformations in which antibodies may bind to vFcγRs: 1) the native conformation of the vFcγR and 2) vFcγR already bound to the Fc region of IgG. First, we will assess Fab binding to native vFcγRs utilizing only the Fab fragment of the antibody. To assess Fab binding to vFcγRs bound to Fc, we will first add Fc from pa-pain digested Cytogam to transfected cells, then add Fab and stain cells to detect Fab binding. Since Cytogam produced from pooled CMV-seropositive plasma, we will also assess IgG responses against vFcγRs in a panel of 30-40 seropositive individuals using the same antibody digestion and staining methods described above to determine the magnitude and frequency of anti-vFcγR responses against each vFcγR on an individual level.

3.2 Assessing the Ability of Infection-Elicited IgG Responses Against vFcγRs to Block IgG Fc Binding.

To determine if the antibodies elicited by natural infection can block vFcγR binding to IgG Fc, we will first add serially diluted Fab from Cytogam and purified IgG from seropositive individuals to vFcγR-transfected cells. We will then add the Fc portion of the digested antibody preparations after conjugating to AF647 using a commercially available kit. This will consider a natural IgG subclass distribution, and we will additionally include a similarly labeled humanized IgG1 (RSV-specific Palivizumab). A lower proportion of triple positive cells (DAPI+ GFP+ AF647+) in CMV-seropositive IgG Fab-treated wells vs seronegative IgG Fab-treated wells will indicate vFcγR-specific IgG can block vFcγR-expressing cells from successfully binding IgG Fc.

3.3 Assessing T Cell Responses Against HCMV vFcγRs in Chronically Infected Individuals.

Peripheral blood mononuclear cell (PBMC) samples from 30-40 CMV-seropositive donors will be purchased from the NY Blood Center. T cell responses against each vFcγR will be assessed via intracellular cytokine staining (ICS), after incubation of the cells with 3 overlapping peptide pools (15-mers overlapping by 11) across each vFcγR gene product, representing possible major histocompatibility complex recognition. pp65 peptide pools will be included as a positive control. Potent responses against certain pools will be deconvoluted for further mapping.

### Statistical Analysis.

Each assay in 3.1 and 3.2 will include two technical replicates and the average of technical replicates will be used for analysis. Data from three independent experiments using Cytogam normalized for transfection efficiency will be compared using ANOVA with Tukey's post hoc procedure to adjust p values for multiple testing in pairwise comparisons following a significant ANOVA. Experiments in 3.1 and 3.3 using individual CMV seropositive donor samples will be run with 2-4 technical replicates, repeated for CVs>30%. The magnitude of responses in CMV seropositive donors will be compared between vFcγRs via repeated-measure ANOVA, and the frequency of responses will be assessed via Fisher's exact test.

### Expected Outcomes.

We expect frequent, low levels of Fab binding to each vFcγR to result from natural HCMV infection, which would be indicated by above background detection of Fab bound to either vFcγR transfected cells or Fc-bound vFcγR transfected cells. However, if we detect no or infrequent antibody binding against any one of the vFcγRs, then that vFcγR may make a better vaccine target than antigens that elicit antibody responses similar to that of natural infection as natural immunity is not protective against re-infection28. Similarly, if antibodies are elicited against each vFcγR as expected, but they do not function to block vFcγR-Fc binding, vaccination with specially designed vFcγR antigens for Fc blocking with optimized adjuvants may be able to elicit such functional antibodies.

### Example 4: Assessing the Humoral and Cellular Immunogenicity of Glycoprotein Subunit Vaccination With and Without Co-Immunization With HCMV vFcγRs

The present example encompasses a hypothesis that addition of vFcγRs to gB and/or PC protein subunit vaccination will elicit robust humoral and cellular responses against the vFcγRs but will not impact responses against gB or PC. We will utilize a step-wise optimization strategy to choose the best combination of glycoprotein target(s) and vFcγR(s) adjuvanted with a squalene emulsion, immunizing rabbits at weeks 0, 4, and 8, with bi-weekly blood collection through week 16. We will measure plasma antibody binding responses over time against each respective vFcγR and glycoprotein as well as neutralization on fibroblasts and epithelial cells. Finally, vaccine-elicited T cell responses will be assessed by ICS using vFcγR and gB peptide.

The ability of co-immunization with HCMV glycoprotein(s) (gB and/or PC) and vFcγR protein(s) to elicit more potent Fc-mediated humoral immune responses against both vaccine components will be tested and non-inferior immunity compared to immunization with glycoprotein(s) alone. To test this, we will vaccinate rabbits, a vaccine immunogenicity model that is optimal for assessment of Fc-mediated antibody functions, with the glycoprotein targets alone or with one or a combination of the vFcγRs as protein subunits with a squalene emulsion adjuvant (Addavax, biosimilar of MF59) and measure antibody and T cell responses.

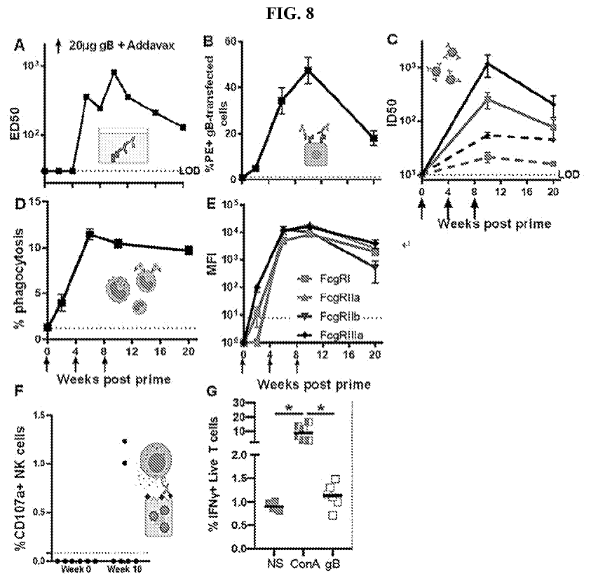

gB/Addavax vaccination elicits gB-specific IgG binding and functional antibody responses. Our previous study defined antibody and T cell responses to a gB subunit vaccine in the rabbit immunogenicity model, FIG. 8. Rabbits were vaccinated with gB protein (Sanofi) lacking only the transmembrane domain, adjuvanted with Addavax at weeks 0, 4, and 8. Similar to observations in gB/MF59 clinical trial vaccinees, gB-specific IgG binding and neutralizing antibody responses peaked two weeks after the last booster dose. Additionally, robust ADCP and gB-specific IgG Fc-binding responses developed in response to vaccination, while ADCC responses were only detectable at low levels in 2/6 animals.

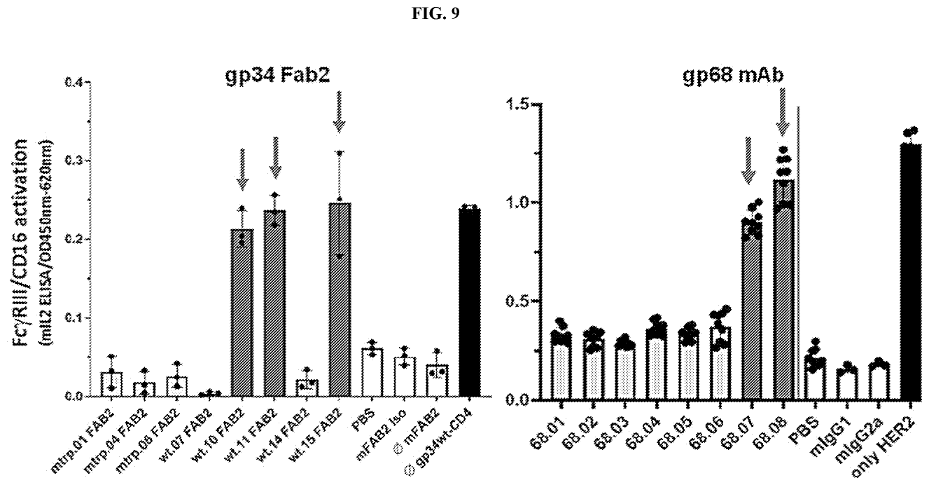

The present example shows that gp34- and gp68-specific monoclonal antibodies (mAbs) can block vFcγR activity and rescue host FcR activation. BALB/c mice were immunized with 50 μg of His-tagged gp34 (wild-type or mtrp mutant lacking Fc binding capability) or gp68 ectodomain in complete Freund's adjuvant with a booster dose of 50 μg in incomplete Freund's adjuvant 2 weeks later. Hybridoma cells were generated from spleen cells and screened for gp34 and gp68 specificity. A subset of gp34- and gp68-specific mAbs were able to rescue CD16 activation in the presence of their respective target, FIG. 9, suggesting that the vFcγR proteins proposed for use as vaccine antigens are immunogenic at a high dose and that a subset of elicited antibodies can block vFcγR function.

Production and quality control of vFcγR protein subunits. The vFcγRs are all type I transmembrane glycoproteins and normally expressed on the surface of infected cells and/or the virion. Thus, we have designed codon-optimized plasmids for the consensus sequence (GenBank) of each vFcγR lacking the transmembrane domain and with a His tag to be used for protein purification. Following amplification and quality control by sequencing, we produced each protein in mammalian 293F cells and purified using nickel-coated beads. We performed Coomassie-stained SDS-PAGE gels as quality control measures for proper protein expression. We further con-firmed IgG Fc binding via ELISA using CMV-seronegative plasma. These results confirmed the size and Fc-binding function of the vFcγR vaccine antigens produced in-house.

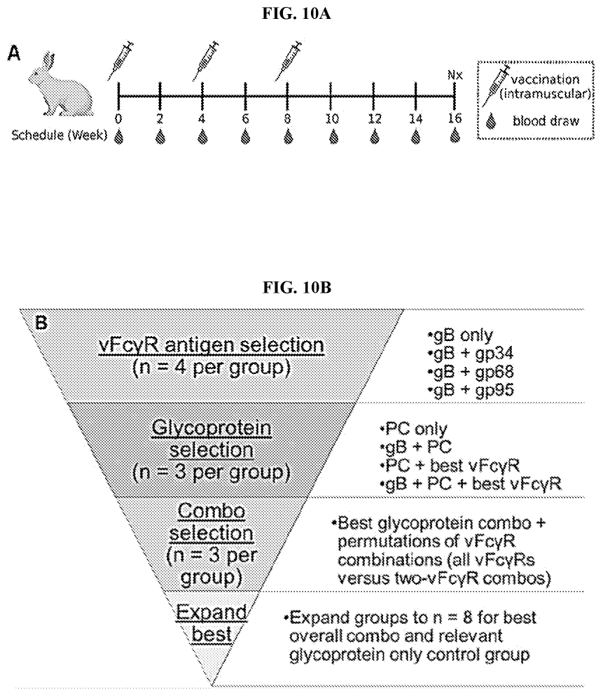

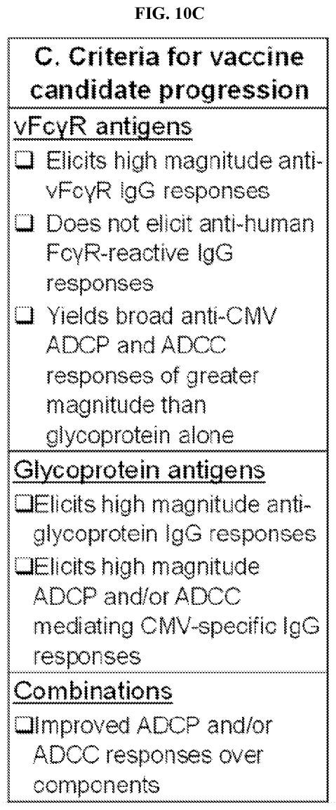

Rabbit vaccine regimen. We have produced large stocks of purified, endotoxin-free vFcγR subunits to use in vaccine formulation. We will vaccinate rabbits with 20 μg of each included protein, which will include combinations of commercially available gB and/or PC protein alone or with one or more soluble vFcγR protein, adjuvanted with commercially available squalene emulsion Addavax, using small group sizes for preliminary assessments of each antigen combination followed by a larger group of the most promising candidate based on preliminary results of the small groups, FIG. 10A-C. Animals will be vaccinated at weeks 0, 4, and 8, with blood draws every two weeks during the study period. At week 16, animals will be necropsied, including terminal blood and lymphoid tissue collection. With the receipt of a National CMV Foundation pilot award, we have been able to initiate groups assessing the immunogenicity of gB+ single vFcγRs, FIG. 10B, grey tier. Thus, this example describes a downselection of gB/PC and gp34/64/95 vaccine components alone and in combination, as well as an expansion of the top-performing antigen combination for statistically-powered comparison of responses between the FcγR vs non-FcγR containing regimen.

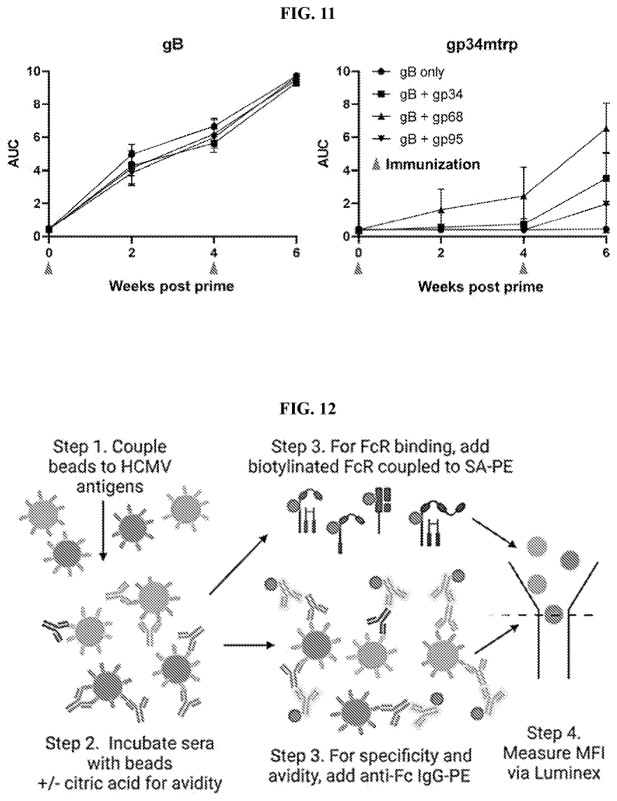

Antibody responses develop against gB and vFcγRs with single co-immunizations. Preliminary results from the pilot study, FIG. 10B, grey tier, demonstrate strong antibody responses through Week 6 of the vaccine regimen, FIG. 11. Strong gB-specific IgG responses develop after the first immunization and continue after boosting. Binding IgG responses measured against vFcγR Fc-binding site mutant gp34mtrp, which is His tagged (gB is not tagged), demonstrates immune responses are also developing against vFcγR antigens.

4.1 Comparing the Humoral Immunogenicity of gB/PC Plus vFcγR Subunit Vaccines to That of gB/PC Alone.

We will first assess antibody binding responses against gB and each respective vFcγR by ELISA as well as plasma neutralization in fibroblasts and epithelial cells using a standard neutralization assay with tropism-matched virus strains. vFcγRs have demonstrated cross reactivity with rabbit IgG1, so antibody binding against vFcγRs must be assessed by Fab only binding, so an aliquot of each plasma sample will be designated for IgG purification and digestion, for this assay, and detection will be performed with anti-rabbit IgG Fab secondary antibody. The magnitude of the gB/PC-specific IgG responses will be compared between gB and each gB/vFcγR co-immunization.

### 4.2: T Cell Responses to Vaccine Antigens.

We will utilize ICS to assess both gB/PC- and vFcγR-specific T cell responses separately over time in rabbit PBMCs and spleen using peptide pools from the respective vaccine antigens as described above. We will detect rabbit lymphocyte markers (pan T cell, CD4, CD8, CD45, CD11b, IgM, IgG) and cytokines (IFNγ, IL-1a, IL-2, IL-4, IL-6, IL-17A, MCP-1) using a panel of biotinylated anti-rabbit antibodies (Bio-Rad) for acquisition on BD Symphony A5. Another non-vaccine HCMV gene peptide pool, such as IE-1, and PMA/ionomycin will be used to generate negative and positive controls, respectively.

### Statistical Analysis.

Downselection of vaccine regimens in small groups will be based on response magnitude only. For comparison of the expanded best glycoprotein+/−vFcγR regimen, we will utilize false discovery rate (FDR) corrected one-way ANOVAs to compare glycoprotein and vFcγR-specific immune responses between groups. Dunnett's post hoc procedure following ANOVA test will be used to adjust p values for multiple testing when comparing each vFcγR vaccine group to the corresponding glycoprotein-only vaccine group.

### Expected Outcomes.

We expect to observe vaccine-elicited gB/PC- and vFcγR-specific antibody and T cell responses, consistent with observations of other squalene adjuvanted subunit vaccines. If antibodies develop against each respective vFcγR, we will not expect to observe differences in elicited gB-binding IgG and neutralizing responses, as vFcγRs are unlikely to play a role in viral entry.

We will also confirm that vFcγR-targeting antibodies do not cross react with host FcγRs. Cross reactivity is unlikely given 1) the lack of amino acid sequence similarity between vFcγRs and host FcγRs, 2) different binding epitopes of IgG Fc, and 3) reliance on Fc glycans for host FcγR binding, which is not the case for vFcγRs. However, we will additionally assess Fab binding to host FcγRs to confirm. If any cross reactivity is observed, the relevant vFcγR will be removed from further consideration as a vaccine antigen.

### Example 5: Defining the Ability of HCMV vFcγR Co-Administration With Glycoprotein Subunit Vaccination to Enhance Fc-Mediated Effector Functions

The present example encompasses a hypothesis that vFcγR vaccine-elicited antibodies will prevent vFcγR-mediated interference with host Fc-mediated effector responses directed against gB and/or PC. We will measure Fc-mediated effector antibody functions, including ADCC and ADCP, against a panel of HCMV strains and expect to see improved Fc mediated antibody effector responses in animals receiving a vFcγR combination vaccine compared to glycoprotein vaccination alone. We will also assess host FcγR binding of vaccine-elicited gB-specific IgG in the presence and absence of vFcγRs in the vaccine.

5.1 Determining the Impact of Inclusion of vFcγRs as a Vaccine Immunogen on Vaccine-Elicited Antibody Fc-Mediated Effector IgG Responses.

We will measure key anti-HCMV Fc-mediated effector responses, ADCP and ADCC, against a panel of HCMV strains with genetic diversity in the vFcγRs, gB, and/or PC (e.g, AD169r, Towne, Toledo, TB40/E, Merlin) and compare the response in rabbits that received glycoprotein only or plus vFcγR vaccines. ADCP will be measured by conjugating purified HCMV virions to a fluorophore (AF647), incubating virions with plasma, and then adding THP-1 monocytes which express CD64 and CD32A. ADCP is reported as the percentage of live AF647+ cells29. ADCC is measured by incubating heat-inactivated plasma with HCMV-infected cells and co-culturing with NK92 cells expressing CD16 or primary NK cells29. A protein transport inhibitor will be used to maintain cell-surface CD107a expression after effector cell activation. Effector cells will be stained for CD107a, CD56, and CD16, and report the percentage of live NK (CD56+) cells expressing CD107a for infected cells minus that of mock infected cells. The infection efficiency is determined by staining a sample of the infected cells to ensure consistency across assays.

If vaccine-elicited ADCC is undetectable, given the low ADCC responses to gB alone (FIG. 8), then we will repeat the ADCC with the vaccinated rabbit plasma plus Cytogam, our positive control for CMV-specific ADCC activity, to determine if the vaccine-elicited antibodies can block vFcγR inhibition of ADCC mediated by Cytogam. As an alternative, we will also utilize an IL-2 reporter cell assay developed by the Hengel lab to evaluate host FcγR activation in the presence or absence of vFcγRs and plasma antibodies from immunized rabbits.

5.2 Assessing Vaccine-Elicited Glycoprotein-Specific IgG Binding to Host FcγRs in the Setting of vFcγR Co-Immunization.

Utilizing a binding antibody multiplex assay (BAMA, FIG. 12), we will measure human FcγRs I, IIA, IIB, and III binding to gB and/or PC-specific IgG in vaccinated rabbits. In brief, FcγR BAMA is performed by coupling antigen(s) to beads with a unique signature that is detectable by the Luminex cytometer. The beads for the antigens of interest are pooled and incubated with plasma samples. Fluorescently labeled FcγRs are added separately for detection of antigen-specific IgG binding by individual FcγRs. FcγR binding is reported as the mean fluorescence intensity (MFI), background subtracted for blank beads.

### Statistical Analysis.

We will utilize the same statistical analysis strategy as Example 4, comparing responses be-tween vFcγR-containing immunizations to the relevant glycoprotein only control. However, the study is not powered to detect differences between the small preliminary groups, so the decision-making criteria outlined in FIG. 11 will be defined by the raw magnitude of observed differences. For the final, large groups we will utilize FDR-corrected Wilcoxon rank-sum tests to determine the effect of vFcγR addition to glycoprotein only vaccina-tion on each response, with ADCP and ADCC as primary endpoints. Based on data from a previous rabbit vac-cine study (10.57±1.34% phagocytosis), 8 animals per group yields 83% power to detect a 20% (2.11% phagocytosis) increase in ADCP responses between the gB only and any of the gB/vFcγR vaccine groups at week 10. ADCC responses were undetectable in 4/6 rabbits vaccinated with gB/Addavax, so even small increases in ADCC response in gB/vFcγR co-vaccination groups will be notable.

All data goes through a rigorous quality control by a blinded member of the lab who did not perform the assays. This involves evaluating flow gating, checking for copy/paste errors from raw data files, ensuring variation between duplicate measurements is below the pre-set threshold (% CV<30), and the performance of positive controls using Levy-Jennings curves. Any samples or assays not meeting QC criteria will be repeated. Further, all statistical analysis will be done in collaboration with an expert biostatistician.

Equal numbers of male and female plasma and PBMC donors in Example 3 and rabbits in Examples 4 and 5 will be analyzed, and thus, responses between the sexes can be compared.

### Expected Outcomes.

We expect improved glycoprotein-specific Fc-mediated antibody effector responses in co-vaccinated animals compared to gB/PC-only-vaccinated animals, but we would not expect to observe a dif-ference in vaccine-elicited IgG host FcγR binding in the presence and absence of co-vFcγR vaccination. We expect to observe a breadth of responses across HCMV strains, with the exception of gp95 co-vaccinations as it is highly polymorphic. If we observe antibody responses against vFcγRs and no differences in vaccine-elicited Fc-mediated effector responses, this suggests that the antibodies elicited against the vFcγRs epitopes are incapable of blocking the vFcγR-Fc interaction. In this case, future studies should employ B cell repertoire studies to better define epitope targets, expected to be at or near the active site of each vFcγR.

The vaccine combination determined through the strategy outlined in FIG. 10A-C will be ideal for moving beyond the initial studies into translation of this novel vaccine approach. Future directions include exploring other vaccine platforms (e.g. mRNA-LNP), and rational design of vFcγR antigens that best display the vFcγR epitopes that can bind to IgGs and block host FcR binding. Once immunogenicity is established and an optimal antigen combination defined, the efficacy of this approach can be studied in the RM model of cCMV infection using the RhCMV homologs of the vaccine antigens selected here.

Vaccination against HCMV is a critical strategy for reducing cCMV-associated neurologic impairment in newborns and including vFcγR(s) in HCMV vaccines may be a solution to the inadequate immunogenicity and protection observed in prior HCMV vaccine trials.

### Example 6: Model Validation

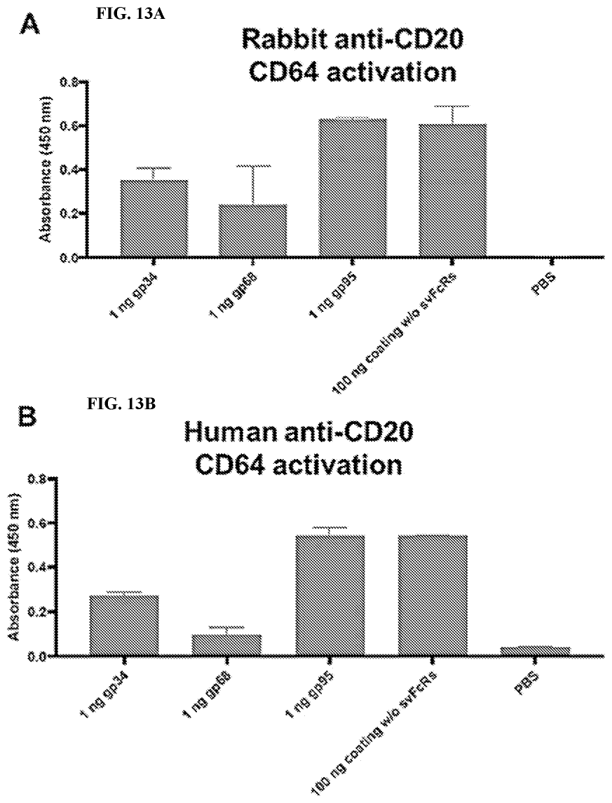

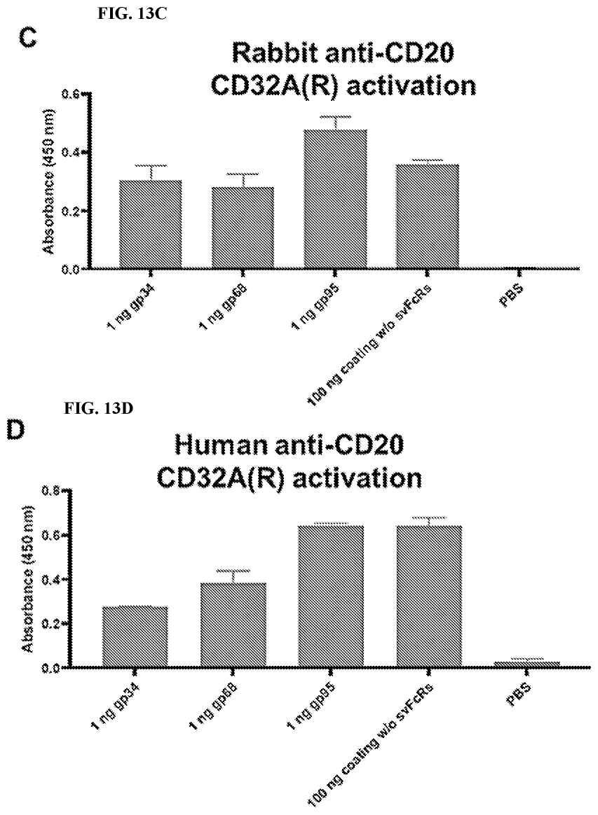

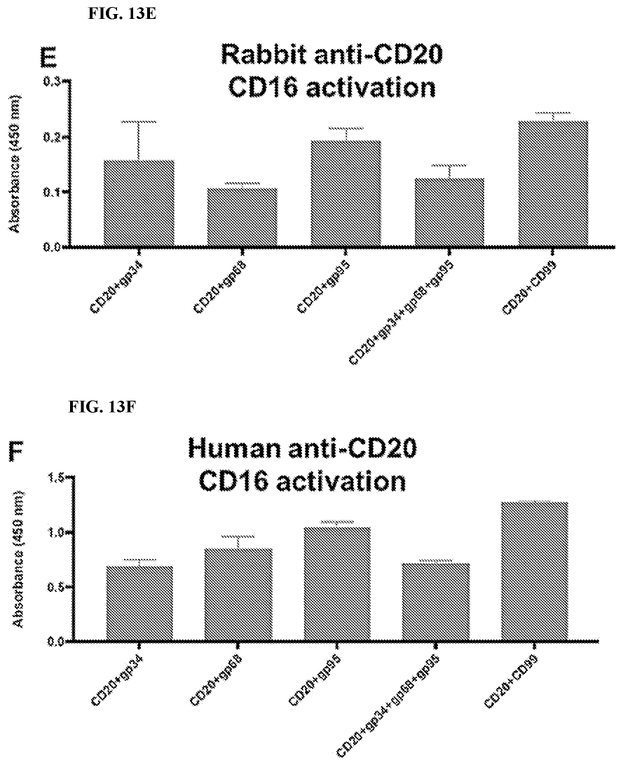

Using an established IL-2 reporter cell assay (Corrales-Aguilar, et al. 2013, DOI: 10.1016/j.jim.2012.09.006), we demonstrate that host CD64 and CD32A activation was reduced compared to conditions without the vFcγRs similarly against immobilized rabbit (FIG. 13A and FIG. 13C) or human (FIG. 13B and FIG. 13D) anti-CD20 when HCMV vFcγRs are introduced as soluble competitors. We note that gp95 appears to be minimally effective in this context, but with a similar pattern observed between the effects on rabbit and human IgG. In a separate experiment using HeLa target cells transfected with CD20 with each vFcγR, all three, or CD99 as a control, we see gp95's impact in reducing CD16 activation (FIG. 13E and FIG. 13F), suggesting that it requires cell surface expression to function. Similarly, the pattern of FcγR activation is similar between rabbit and human IgG. Further work is ongoing to fully characterize the impact of HCMV vFcγRs in human FcγR activation by rabbit IgG

### Example 7: Immunogenicity

Rabbits were immunized with 20 μg gB alone or with 20 μg or 40 μg individual vFcγRs (gp34, gp68, or gp95) adjuvanted 1:1 with Addavax. We conducted a study to assess the effect of dose on immunogenicity, comparing additional groups receiving 40 μg of each vFcγR. This indicates that all three vFcγRs are immunogenic at the high dose, resulting in improved Fc mediated immune function.

7.1 vFcγR-Binding Responses.

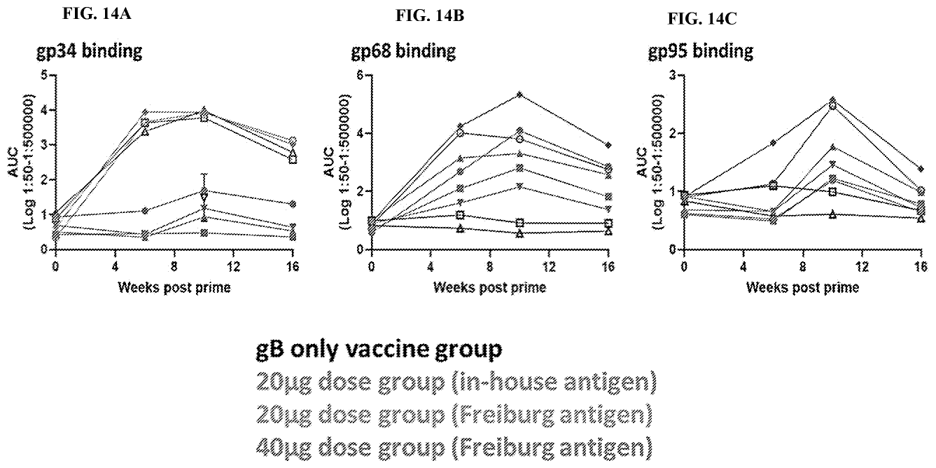

We initially measured binding against each vFcγR within the rabbits receiving each respective vFcγR-containing vaccine via ELISA to assess binding of whole IgG in rabbit plasma (FIG. 14A-FIG. 14C). These results demonstrate that antibody responses develop against the vFcγRs, with the strongest responses generally observed with the 40 μg dose groups. We plan to validate these results using Fab-only detection of binding to eliminate the background from vFcγR-Fc interactions. Binding results against gp34 suggest differences in the immunogenicity of different antigen preparations, and we will further investigate potential reasons for this discrepancy.

7.2 Inclusion of HCMV vFcγRs at 40 μg Dose in gB Protein Subunit Vaccine Improves ADCP Function Broadly Across Several HCMV Isolates.

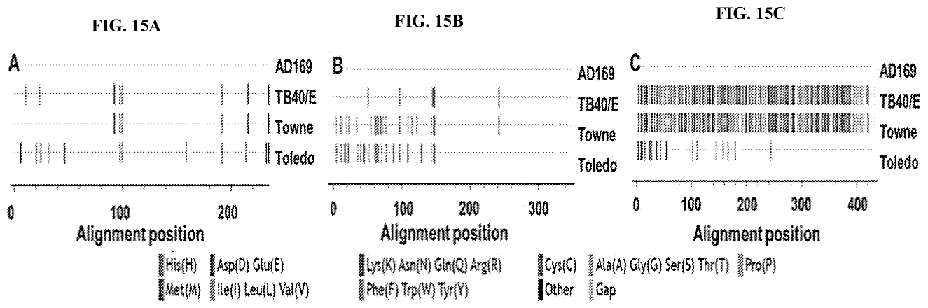

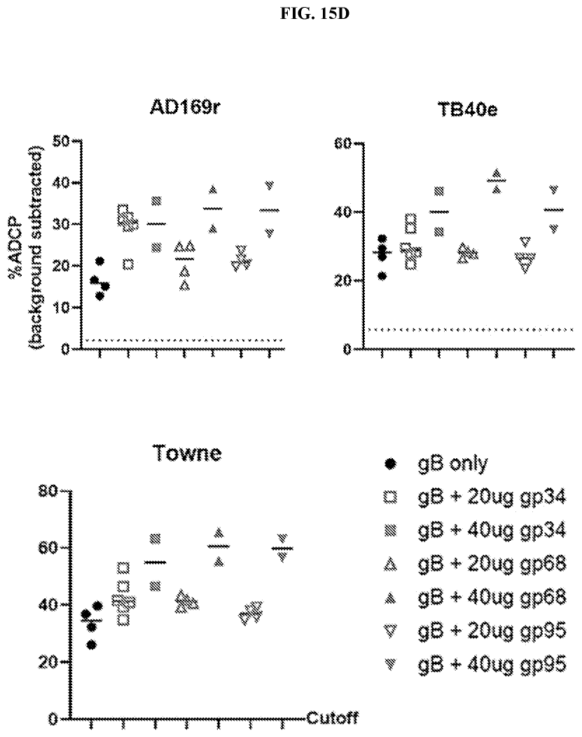

Highlighter plots of (FIG. 15A) RL11 encoding gp34, (FIG. 15B) UL119-118 encoding gp68, and (FIG. 15C) RL12 encoding gp95 from select HCMV isolates demonstrate strong conservation across RL11, moderate conservation of UL119-118, and high variability of RL12 potentially suggesting two isoforms. Using peak immunity at week 10 post prime, we show that antibody dependent cellular phagocytosis (ADCP) function was significantly greater compared to the gB only baseline with the addition of 20 μg gp34, but gp68 and gp95 did not demonstrate clear improvement at the 20 μg dose (FIG. 15D). Animals receiving 40 μg doses of each vFcγR demonstrate greater ADCP function across a variety of HCMV strains with variations in vFcγR sequences.

7.3 ADCC Function Detectable in a Greater Number of Animals Immunized With vFcγRs Compared to gB Alone.

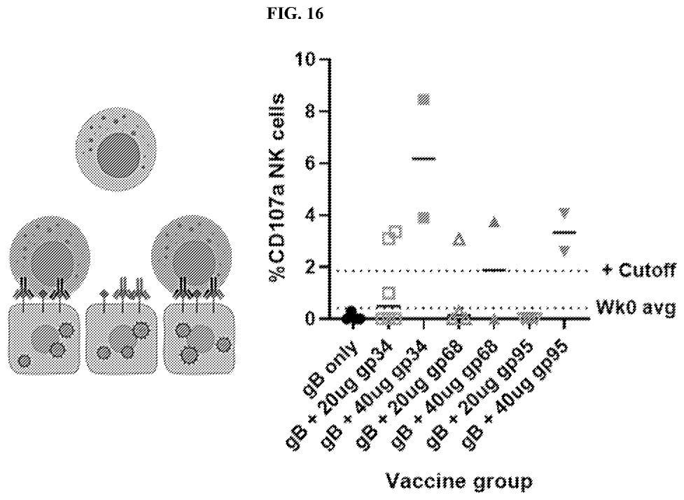

We measured ADCC function via CD107a+ NK cells from primary PBMCs after exposure to HCMV-infected fibroblasts with rabbit plasma at week 10 post prime (FIG. 16). ADCC function is undetectable in all animals in the gB only vaccine group but above the positivity cutoff in several animals immunized with gB in combination with each vFcγR, including both animals in the 40 μg dose group for gp34 and gp95.

### Example 8: Optimizing Antigen Combinations

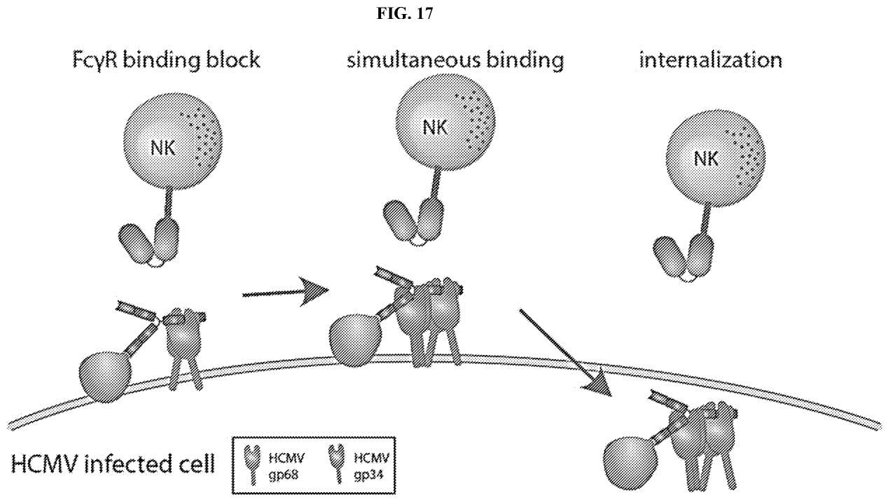

vFcγRs are not redundant (FIG. 17). In Kolb et al. (eLife 2021 doi:10.7554/eLife.63877) we identify the binding regions of HCMV vFcγRs on IgG and show that NK cell mediated ADCC is antagonized by non-redundant, cooperative mechanisms elicited by simultaneously binding gp68 and gp34 (natively a dimer). gp68 binds IgG in a 2:1 ratio reducing, but not abolishing accessibility of immune complexes to host FcγR immune effector cells such as NK cells. gp34 effectively internalizes immune complexes making them unavailable to surveilling FcγR effector cells but cannot compete with host FcγRs for a similar binding region on IgG. The mechanism by which gp95 interferes with Fc-mediated immunity is currently unknown.

Since the vFcγRs function by distinct and non-redundant mechanisms, we next plan to evaluate combinations of vFcγRs together an in combination with other CMV base antigens besides gB, including the pentameric complex, UL16, and UL141. We further aim to assess key epitope targets to elicit functional blocking of vFcγRs to aid in antigen design to optimize immune responses toward maximizing Fc mediated effector functions.

Due to the high species specificity of CMVs, we cannot evaluate efficacy in the rabbit model, so we plan to assess efficacy in protection from initial infection and vertical transmission in a rhesus macaque model, adapting either the vaccine antigens to the rhesus CMV homologs or developing a challenge rhesus CMV containing the human CMV vFcγRs in place of their respective homologs.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An immunogenic composition comprising one or more cytomegalovirus (CMV) vFcγRs antigens comprising gp34, gp68, gp95, or an immunogenic fragment of any thereof.

2. A combination comprising two or more cytomegalovirus (CMV) vFcγRs antigens comprising gp34, gp68, gp95, or an immunogenic fragment of any thereof. A combination comprising: (a) one or more cytomegalovirus (CMV) vFcγRs antigens or a nucleic acid encoding the same, and (b) one or more additional CMV antigens or a nucleic acid encoding the same, wherein the vFcγRs antigens comprise gp34, gp68, gp95, or an immunogenic fragment of any thereof, and wherein the additional CMV antigens are different from the vFcγRs antigens.

4. The combination of claim 3, wherein the one or more additional CMV antigens comprise glycoprotein B (gB), pentameric complex (PC), pp65, immediate-early protein-1 (IE-1), or an immunogenic fragment of any thereof.

5. The combination of any one of claims 2-4, wherein the one or more CMV vFcγRs antigens are polypeptides.

6. The combination of any one of claims 3-5, wherein the one or more additional CMV antigens are polypeptides.

7. The combination of any one of claim 2-4, comprising a nucleic acid encoding the one or more CMV vFcγRs antigens.

8. The combination of claim 3, 4, 6, or 7, comprising a nucleic acid encoding the one or more additional CMV antigens.

9. The combination of any one of claims 2-8, wherein the one or more CMV vFcγRs antigens comprise gp34 or an immunogenic fragment thereof.

10. The combination of claim 9, wherein the one or more CMV vFcγRs antigens further gp68 or an immunogenic fragment thereof.

11. The combination of claim 9 or 10, wherein the one or more CMV vFcγRs antigens further gp95 or an immunogenic fragment thereof.

12. The combination of any one of claims 2-11, wherein the one or more CMV vFcγRs antigens can elicit an antibody response that blocks vFcγR binding to IgG Fc in a subject.

13. The combination of any one of claims 3-12, wherein the one or more additional CMV antigens comprise (i) gB or an immunogenic fragment thereof and (ii) PC or an immunogenic fragment thereof.

14. A combination comprising a gp34 polypeptide or an immunogenic fragment thereof and a glycoprotein B (gB) polypeptide or an immunogenic fragment thereof.

15. The combination of claim 14, further comprising a gp68 polypeptide or an immunogenic fragment thereof.

16. The combination of claim 14 or 15, further comprising a gp95 polypeptide or an immunogenic fragment thereof.

17. A vaccine comprising the immunogenic composition of claim 1 or the combination of any one of claims 2-16.

18. The vaccine of claim 17, further comprising an adjuvant.

19. The vaccine of claim 18, wherein the adjuvant is MF59, Addavax, or other squalene emulsion adjuvant.

20. A pharmaceutical composition comprising the combinations of any one of claims 2-16.

21. A method comprising administering one or more doses of the combination of any one of claims 2-16 to a subject.

22. The method of claim 21, wherein the method induces an immune response in a subject.

23. The method of claim 21 or 22, wherein the subject is a female of child-bearing age.

24. The method of claim 21 or 22, wherein the subject is at risk for severe CMV disease.

25. The method of claim 24, wherein the subject has cancer, HIV, and/or has received a solid organ transplant.

26. A method of any one of claims 21-25, wherein the administering generates anti-CMV antibodies in a subject.

27. The method of any one of claims 21-26, wherein the administering has improved Fc-mediated antibody effector response to the one or more additional CMV antigens, relative to a subject that has been administered only the one or more additional CMV antigens.

28. The method of any one of claims 21-27, wherein the administering has increased CD64 activation, increased CD16 activation, and/or increased CD32 activation, relative to a subject that has been administered only the one or more additional CMV antigens.

29. The method of any one of claims 21-28, wherein the administering has increased antibody dependent cellular cytotoxicity (ADCC) and/or increased antibody-dependent cellular phagocytosis (ADCP) response, relative to a subject that has been administered only the one or more additional CMV antigens.
